(19) United States
(12) Patent Application Publication
(10) Pub. No.: US 2025/0041557 A1
(43) Pub. Date: Feb. 6, 2025

(54) COMPUTERIZED ELECTRONIC ENVIRONMENTAL CONTROL SYSTEM AND METHODS THEREOF

(71) Applicant: PLUME DESIGN, INC., Palo Alto, CA (US)

(72) Inventors: Bethany YEATON, Postmouth, NH (US); Kanagavel RAVINDRAN, V, Sunnyvale, CA (US); Chinliang KO, Taipei (TW)

(21) Appl. No.: 18/365,159

(22) Filed: Aug. 3, 2023

(51) Int. Cl. A61M 21/02; A61B 5/00; A61B 5/11

(52) U.S. Cl. CPC A61M 21/02; A61B 5/1113; A61B 5/4806; A61M 2021/0044; A61M 2021/0066; A61M 2021/0083

(57) ABSTRACT

Disclosed are systems and methods that provide a novel framework for personalized environmental control to optimize user sleep and wakefulness. The framework can include dynamically controlling devices within a user's home environment based on a computerized bedtime or waketime schedule. The disclosed framework can operate to temporally modify an location-based environment for a user, which can correspond to how devices function, whether they are on/off, whether they are accessible (e.g., turned on or off), and the like. Accordingly, the framework can automatically control how devices operate a location based on activity patterns of a user so as to enable peak performance of particular activities (e.g., better sleep due to device thresholding).

### FIELD OF THE DISCLOSURE

The present disclosure is generally related to an environmental control system, and more particularly, to a computerized framework for computer-generated, temporally and/or contextually based schedules that control real-world and/or digital stimuli in a location.

### BACKGROUND

In an age in which sleep tracking devices are widely available, many individuals seek informative measures of their sleep quality. However, conventional devices, systems and applications fail in helping their users to physically and/or digitally implement changes to procedures and/or locations' environments that could improve their sleep.

### SUMMARY OF THE DISCLOSURE

The disclosed systems and methods, as described herein, provide a novel computerized framework that (1) dynamically determines a routine for a user, which can correlate to digital, networking and/or physical activities at and/or around a location(s), from which can be leveraged to (2) dynamically control, manage and/or modify a location's environment (e.g., home) of the user. As discussed herein, such novel, computerized functionality can aide in preparing the user for an activity (e.g., prepare for sleep and/or wake), as well as prepare the location to aide in such activity (e.g., adjust sound, light, temperature, humidity, and the like in at least the bedroom of the user in advance of their sleep). Indeed, according to some embodiments as discussed infra, the disclosed computerized framework can be implemented for routines corresponding to when a user(s) is going to sleep, during sleep (e.g., at specific onsets of types of sleep (e.g., REM cycle, for example)), in advance of their wake from sleep, and the like, or some combination thereof.

According to some embodiments, the techniques described herein relate to a computer-implemented method including (1) generating, for a user based on data collected by one or more sensors in a home environment of the user, a user bedtime schedule including an action that the user is predicted to perform in preparation for sleep, (2) identifying a device associated with the action and an adjustment to a stimulus controlled by the device that is associated with an improved measure of sleep, and (3) in response to determining that the user is performing the action at a moment in time in preparation for sleep, or that the user is predicted to perform the action in preparation for sleep at the moment in time, instructing the device to apply the adjustment to the stimulus at the moment in time. In some aspects, the method further includes triggering the one or more sensors to collect the data. In some aspects, the method further includes inputting the data collected by the one or more sensors to a computerized model. In these aspects, identifying the device and the adjustment associated with the improved measure of sleep may include receiving at least one of the device or the adjustment as an output from the computerized model. Additionally or alternatively, generating the bedtime schedule can include receiving the bedtime schedule as an output from a computerized model.

In some aspects, the device represents or controls a light source and the stimulus represents a light emitted from the light source with an adjustable color temperature or brightness. In some aspects, the device controls an adjustable temperature in the home environment, or controls an additional device that controls the adjustable temperature, and the stimulus represents the adjustable temperature. In some aspects, the device controls Internet traffic within the home environment, and/or presents content received via the Internet traffic, and the stimulus represents the content received via the Internet traffic (e.g., any content received via the Internet traffic or particular content received via the Internet traffic). In some aspects, the one or more sensors include sensor capabilities for tracking movements and information about the user at the home environment.

In some aspects, the techniques described herein relate to a method including (1) generating, for a user based on data collected by one or more sensors in a home environment of the user, a user waketime schedule including a time at which the user is expected to wake up, (2) identifying an adjustment to a stimulus controlled by a device that is associated with improved wakefulness, and (3) in response to identifying the adjustment associated with improved wakefulness, instructing the device that controls the adjustment associated with improved wakefulness to apply the adjustment associated with improved wakefulness at the time at which the user is predicted to wake up. In some aspects, the waketime schedule further includes an additional action that that user is expected to perform at an additional moment in time after waking up, the method further includes identifying a device associated with the additional action and an adjustment to a stimulus, controlled by the device associated with the additional action, that is associated with improved wakefulness, and the method further includes, in response to determining that the user is performing the additional action at the additional moment in time, or that the user is predicted to perform the additional action at the additional moment in time, instructing the device associated with the additional action to apply the adjustment associated with improved wakefulness at the additional moment in time. Each of these aspects of the disclosed framework will be discussed in more detail below in relation to at least FIGS. 1 and 3-4.

It should be understood that while the discussion herein may be focused on a home being a location, it should not be construed as limiting, as any other type of location can be utilized as a basis for the purposes of the disclosed systems and methods without departing from the scope of the instant disclosure. For example, a location, in addition to a user's home, can be, but is not limited to, a hotel room, another person's home, and the like, or an office or other location for which a user may require/request control of their environment to perform an activity.

According to some embodiments, a method is disclosed for a decision intelligence (DI)-based computerized framework (e.g., for performing one or more of the steps discussed in connection with FIGS. 3-4). In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method for automatically and dynamically managing real-time, time-based (e.g., bedtime/waketime) activity of a user.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary workflow according to some embodiments of the present disclosure;

FIG. 4 also illustrates an exemplary workflow according to some embodiments of the present disclosure;

FIG. 5 depicts an exemplary implementation of an architecture according to some embodiments of the present disclosure;

FIG. 6 depicts an exemplary implementation of an architecture according to some embodiments of the present disclosure; and

FIG. 7 is a block diagram illustrating a computing device showing an example of a client or server device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, a smart ring, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

Certain embodiments and principles will be discussed in more detail with reference to the figures. According to some embodiments, as discussed in more detail below, the disclosed framework can function/operate to provide personalized insights and/or recommendations to users based on (1) their own specific/unique data and/or (2) how their own specific/unique data compares to aggregated data for a population of the user. Thus, a tailored approach to sleep insight and/or improvement can be compiled and automatically provided to the user, which can be provided upon request and/or a determined time (e.g., a time a user is deemed to be about to sleep and/or to have just woken up). In some embodiments, the disclosed framework can leverage such insights, in addition to the recommendations to the users, to control the location in which they are sleeping. For example, access points, devices (e.g., televisions, smart phones, and the like), lights and/or any other smart home feature can be engaged and/or toggled to different and/or modified modes upon the determination of a user's sleep, which can assist the user from engaging in activity that may be detrimental to their known sleep habits.

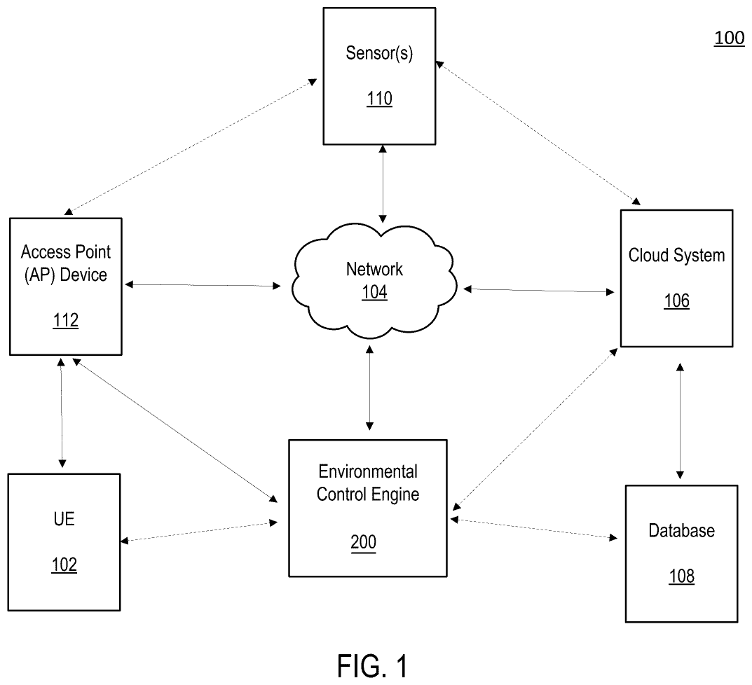

With reference to FIG. 1, system 100 is depicted which includes user equipment (UE) 102 (e.g., a client device, as mentioned above and discussed below in relation to FIG. 4), access point (AP) device 112, network 104, cloud system 106, database 108, sensors 110 and environmental control engine 200. It should be understood that while system 100 is depicted as including such components, it should not be construed as limiting, as one of ordinary skill in the art would readily understand that varying numbers of UEs, AP devices, peripheral devices, sensors, cloud systems, databases and networks can be utilized; however, for purposes of explanation, system 100 is discussed in relation to the example depiction in FIG. 1.

According to some embodiments, UE 102 can be any type of device, such as, but not limited to, a mobile phone, tablet, laptop, sensor, IoT device, autonomous machine, and any other device equipped with a cellular or wireless or wired transceiver. For example, UE 102 can be a smart ring, which as discussed below in more detail, can enable the identification and/or collection of vitals of the wearing user. In some embodiments, such vitals can correspond to, but not be limited to, heart rate, heart rate variability (HRV), blood oxygen levels, blood pressure, hydration temperature, pulse, motion, sleep, and/or any other type of biometric for a person, or some combination thereof.

In some embodiments, a peripheral device (not shown) can be connected to UE 102, and can be any type of peripheral device, such as, but not limited to, a wearable device (e.g., smart ring or smart watch), printer, speaker, sensor, and the like. In some embodiments, the peripheral device can be any type of device that is connectable to UE 102 via any type of known or to be known pairing mechanism, including, but not limited to, WiFi, Bluetooth™, Bluetooth Low Energy (BLE), NFC, and the like. For example, the peripheral device can be a smart ring that connectively pairs with UE 102, which is a user's smart phone.

According to some embodiments, AP device 112 is a device that creates a wireless local area network (WLAN) for the location. According to some embodiments, the AP device 112 can be, but is not limited to, a router, switch, hub and/or any other type of network hardware that can project a WiFi signal to a designated area. For example, an AP device 112 can be a Plume Pod™, and the like. In some embodiments, UE 102 may be an AP device.

According to some embodiments, sensors 110 (or sensor devices 110) can correspond to any type of device, component and/or sensor associated with a location of system 100 (referred to, collectively, as “sensors”). In some embodiments, the sensors 110 can be any type of device that is capable of sensing and capturing data/metadata related to a user and/or activity of the location. For example, the sensors 110 can include, but not be limited to, cameras, motion detectors, door and window contacts, heat and smoke detectors, passive infrared (PIR) sensors, time-of-flight (ToF) sensors, and the like.

In some embodiments, the sensors 110 can be associated with devices associated with the location of system 100, such as, for example, lights, smart locks, garage doors, smart appliances (e.g., thermostat, refrigerator, television, personal assistants (e.g., Alexa®, Nest®, for example)), smart rings, smart phones, smart watches or other wearables, tablets, personal computers, and the like, and some combination thereof. For example, the sensors 110 can include the sensors on UE 102 (e.g., smart phone) and/or peripheral device (e.g., a paired smart watch). In another example, sensors 110 can correspond to the sensors on a user's smart ring.

In some embodiments, network 104 can be any type of network, such as, but not limited to, a wireless network, cellular network, the Internet, and the like (as discussed above). Network 104 facilitates connectivity of the components of system 100, as illustrated in FIG. 1.

According to some embodiments, cloud system 106 may be any type of cloud operating platform and/or network-based system upon which applications, operations, and/or other forms of network resources may be located. For example, system 106 may be a service provider and/or network provider from where services and/or applications may be accessed, sourced or executed from. For example, system 106 can represent the cloud-based architecture associated with a smart home or network provider, which has associated network resources hosted on the internet or private network (e.g., network 104), which enables (via engine 200) the location-based environmental management and control discussed herein.

In some embodiments, cloud system 106 may include a server(s) and/or a database of information which is accessible over network 104. In some embodiments, a database 108 of cloud system 106 may store a dataset of data and metadata associated with local and/or network information related to a user(s) of the components of system 100 and/or each of the components of system 100 (e.g., UE 102, AP device 112, sensors 110, and the services and applications provided by cloud system 106 and/or environmental control engine 200).

In some embodiments, for example, cloud system 106 can provide a private/proprietary management platform, whereby engine 200, discussed infra, corresponds to the novel functionality system 106 enables, hosts and provides to a network 104 and other devices/platforms operating thereon.

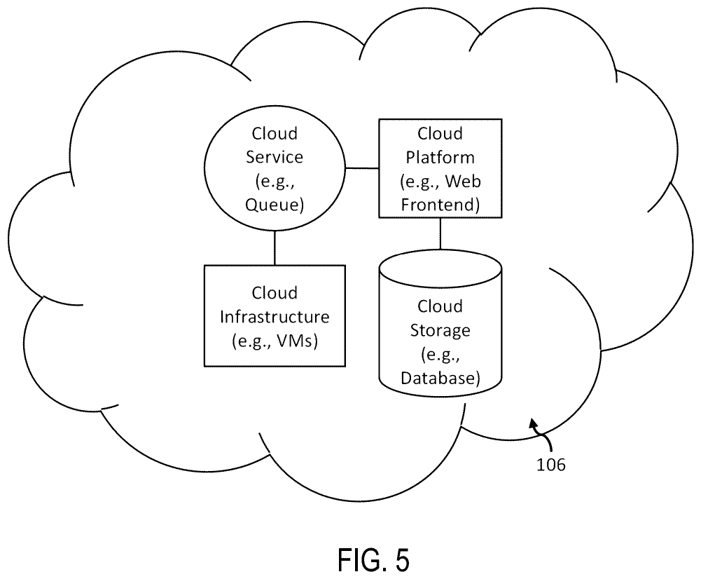

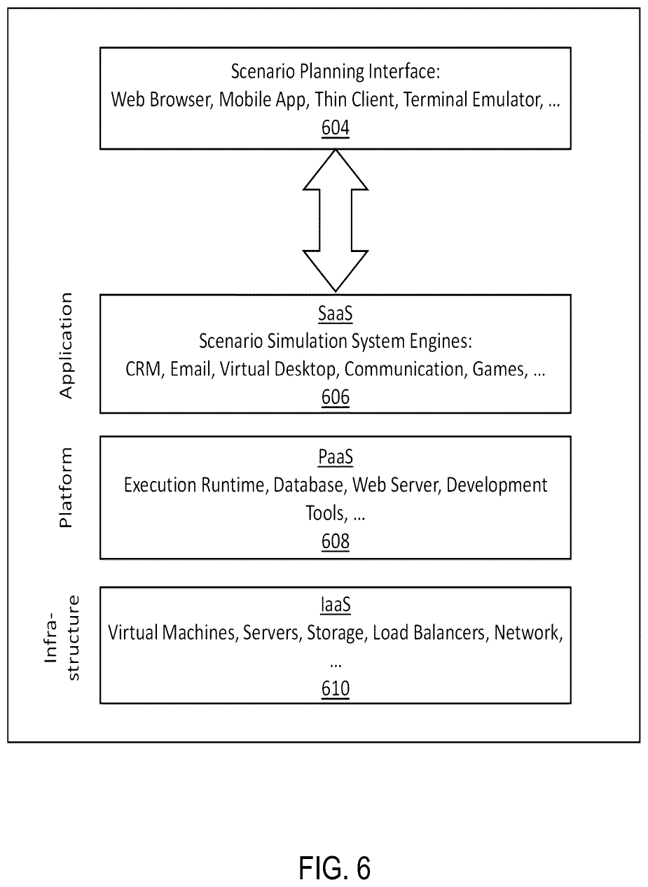

Turning to FIGS. 5 and 6, in some embodiments, the exemplary computer-based systems/platforms, the exemplary computer-based devices, and/or the exemplary computer-based components of the present disclosure may be specifically configured to operate in a cloud computing/architecture 120 such as, but not limiting to: infrastructure as a service (IaaS) 610, platform as a service (PaaS) 608, and/or software as a service (SaaS) 606 using a web browser, mobile app, thin client, terminal emulator or other endpoint 604. FIGS. 5 and 6 illustrate schematics of non-limiting implementations of the cloud computing/architecture(s) in which the exemplary computer-based systems for administrative customizations and control of network-hosted application program interfaces (APIs) of the present disclosure may be specifically configured to operate.

Turning back to FIG. 1, according to some embodiments, database 108 may correspond to a data storage for a platform (e.g., a network hosted platform, such as cloud system 106, as discussed supra) or a plurality of platforms. Database 108 may receive storage instructions/requests from, for example, engine 200 (and associated microservices), which may be in any type of known or to be known format, such as, for example, standard query language (SQL). According to some embodiments, database 108 may correspond to any type of known or to be known storage, for example, a memory or memory stack of a device, a distributed ledger of a distributed network (e.g., blockchain, for example), a look-up table (LUT), and/or any other type of secure data repository

Environmental control engine 200, as discussed above and further below in more detail, can include components for the disclosed functionality. According to some embodiments, environmental control engine 200 may be a special purpose machine or processor and can be hosted by a device on network 104, within cloud system 106, on AP device 112 and/or on UE 102. In some embodiments, engine 200 may be hosted by a server and/or set of servers associated with cloud system 106.

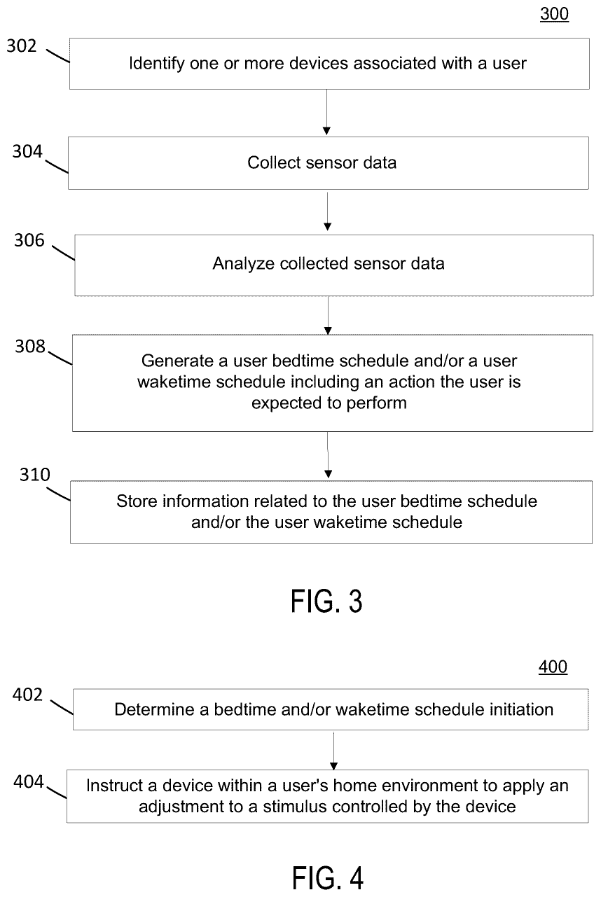

According to some embodiments, as discussed in more detail below, environmental control engine 200 may be configured to implement and/or control a plurality of services and/or microservices. In some examples, each of the plurality of services/microservices are configured to execute a plurality of workflows associated with performing the disclosed environmental control (e.g., in relation to sleep and/or wakefulness optimization management). Non-limiting embodiments of such workflows are provided below in relation to at least FIGS. 3-4.

According to some embodiments, as discussed above, environmental control engine 200 may function as an application provided by cloud system 106. In some embodiments, environmental control engine 200 may function as an application installed on a server(s), network location and/or other type of network resource associated with system 106. In some embodiments, environmental control engine 200 may function as an application installed and/or executing on UE 102 and/or sensors 110 (and/or AP device 112, in some embodiments). In some embodiments, such application may be a web-based application accessed by AP device 112, UE 102 and/or devices associated with sensors 110 over network 104 from cloud system 106. In some embodiments, environmental control engine 200 may be configured and/or installed as an augmenting script, program or application (e.g., a plug-in or extension) to another application or program provided by cloud system 106 and/or executing on AP device 112, UE 102 and/or sensors 110.

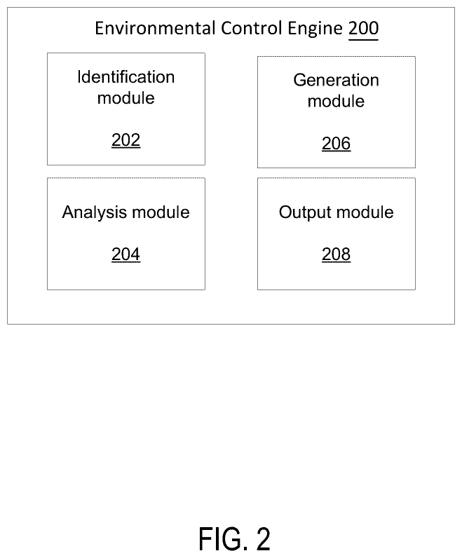

As illustrated in FIG. 2, according to some embodiments, environmental control engine 200 includes identification module 202, analysis module 204, generation module 206 and output module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

Turning to FIG. 3, Process 300 provides non-limiting example embodiments for the disclosed computerized sleep management framework. According to some embodiments, Process 300 provides non-limiting embodiments for determining user information and corresponding patterns of behavior (e.g., a user bedtime routine and/or waketime routine) around which the disclosed system (e.g., via environmental control engine 200) can control, manage and manipulate the operational status of a location. As discussed below at least in relation to Process 400 of FIG. 4, the leveraged and stored information can be utilized to optimize a user's sleep and/or a user's wakefulness.

According to some embodiments, Steps 302-304 of Process 300 can be performed by identification module 202 of environmental control engine 200; Step 306 can be performed by analysis module 204; Step 308 can be performed by generation module 206; and Steps 310 and 312 can be performed by output module 208.

According to some embodiments, Process 300 begins with Step 302 where a set of devices associated with a user (and in some embodiments, associated with a location, for example, a user's home environment) are identified. According to some embodiments, the devices can be associated with any type of UE 102, AP device 112, sensors 110, and the like, discussed above in relation to FIG. 1. For example, the devices can at least include, but are not limited to, a sleep-tracking device (e.g., a wearable device such as a user's smart watch and/or smart ring that they wear daily), the user's smart phone, tablet devices in the home connected to the local network (e.g., Wi-Fi), televisions, light sensors, door sensors, routers/modems providing the network for the location, and the like. Additional, non-limiting examples of sensors and the types of collectable data are discussed above at least in relation to FIG. 1.

In some embodiments, the identified devices can be paired and/or connected with another device (e.g., sensor 110, engine 200 and/or UE 102) via a cloud (e.g., Plume® cloud, for example) and/or cloud-to-cloud (C2C) connection (e.g., establish connection with a third-party cloud, which connects with cloud system 106, for example).

In Step 304, environmental control engine 200 can operate to trigger the identified devices to collect data about the user (e.g., referred to as user data). The user data may be collected as part of monitoring the user's location (e.g., home environment) for data to be used to generate (e.g., determine) a bedtime schedule and/or a waketime schedule for the user. This user data can include any type or form of data relating to a user and/or an environment (e.g., a location and/or dwelling) of the user (e.g., any data that can be collected from a device identified in Step 302). In some examples, the user data can represent or include sensor data (e.g., collected via sensor(s) 110). According to some embodiments, the user data can be collected continuously and/or according to a predetermined period of time or interval. For example, environmental control engine 200 can periodically ping each or a portion of the devices for a reply and/or can push and/or fetch protocols to collect user data from each sensor. Additionally or alternatively, the user data may be collected based on detected events. In some embodiments, a type and/or quantity of user data collected may be directly tied to the type of device performing such data collection. For example, a sensor associated with lights within the user's bedroom may only collect user data when a light in that room is turned on (and/or turned off from previously being turned on). In this example, user data can provide, but is not limited to, an on event, which can indicate, but is not limited to, the identity of the light/room, time of toggling, duration of being turned on, frequency of being turned on, and the like, or some combination thereof. In another non-limiting example, a gyroscope sensor on a user's smartphone and/or smart ring can detect when a user is moving, the type and/or metrics of such movements, etc. In some embodiments, the data collection can involve push and/or fetch protocols to collect user data from each sensor.

According to some embodiments, the user data can be generated (e.g., derived) from data collected from UE 102. For example, as discussed above (e.g., in examples in which user data is collected from a wearable device such as a smart ring), the user data can provide biometrics (e.g., vitals) for the user, which can include information related to, but are not limited to, heart rate, HRV, blood oxygen levels, blood pressure, hydration, body temperature, pulse, motion, and the like, or some combination thereof. In some such examples, the biometrics, inclusive of the heart rate, HRV, blood pressure and blood oxygen level information, for example, can provide insights into a user's cardiovascular health, respiratory health, and/or fitness level. Additionally or alternatively, the biometrics can further provide or contribute to tracked information related to the user's physical activity, including, but not limited to, the number of steps taken, distance traveled, and calories burned, and the like. As another example, the biometrics can be used to derive data relating to the user's stress levels and/or to detect potential illness.

In some examples, the user data can include sleep data (e.g., biometric data, associated with sleep, collected by a sensor of a sleep-tracking device operating with UE 102). In these examples, environmental control engine 200 may detect, for a user, a measure of the user's sleep generated (e.g., derived) based on the sleep data (e.g., by triggering a sensor to collect the sleep data and then generating the measure of the user's sleep based on the sleep data). The measure of the user's sleep can include one or more of any type or form of measurement (value or metric) related to sleep (e.g., sleep quantity and/or quality). Measures relating to sleep can include, but are not limited to, measures relating to sleep latency (e.g., time it takes for the user to fall asleep or into a deep sleep, and the like), total sleep duration, deep sleep duration (e.g., length of time spent in restorative sleep), light sleep duration (e.g., time spent in lighter sleep stages), restlessness (e.g., frequency and/or degree of movement during sleep and/or when lying in bed), nighttime awakenings (e.g., number of times the user wakes up and/or gets out of bed), and the like or some combination thereof.

In addition to collecting user sleep data, in some examples the devices identified at Step 302 may collect sleep-relevant data. Sleep-relevant data may represent data that is not indicative of an amount, type, and/or quality of sleep but that may be associated with (e.g., may influence) the amount, type, and/or quality of sleep experienced by a user. Examples of sleep-relevant data may include, without limitation, environmental data (e.g., data relating to a location such as a home environment and/or a geographic area associated with a user), data relating to a user schedule such as a bedtime and/or waketime schedule, a user's pattern of activity (e.g., a type of activity engaged in by a user and/or a frequency and/or timing of an activity), and/or data relating to a biometric. In some embodiments, the sleep-relevant data can be based on lifestyle factors (e.g., factors inferred from sensor data collected from sensors 110 and/or UE 102). Such lifestyle factors can include, but are not limited to, screen/Internet use (e.g., time spent on screens before bed and/or throughout the day), type of network activity (e.g., gaming, social media, streaming, and the like), exercise patterns (e.g., timing, duration, and/or intensity of physical activity during the day), motion detection and/or user location (e.g., motion data in/around and/or outside the location during the day and/or around bedtime), diet and nutrition, and the like, or some combination thereof. In some examples, all data that may be collected by the devices identified at Step 302 can be considered (e.g., designated) as potentially sleep-relevant. In one such example, any (e.g., all) data collected by such devices may be inputted to a computerized model (e.g., as part of the analyzing that will be described shortly in connection with step 306) and sleep-relevant data may be received as an output from the computerized (e.g., trained) model.

In some embodiments, the user data collected by the devices may include user information submitted by the user. Such information may include, without limitation, a bedtime indicated by the user, a waketime indicated by the user, one or more actions performed by the user as part of a bedtime schedule or waketime schedule (e.g., including, in some examples, timing information for such actions), social information (e.g., contacts submitted by the user), health information (e.g., a health condition submitted by the user), user activity reported by the user (e.g., a submission of foods consumed by the user), a mental health status (e.g., a submission of a level of stress indicated by the user), height information, weight information, BMI information, etc.

The disclosed sleep management system may enable a user to submit user information in a variety of ways. As one example, environmental control engine 200 can (1) generate a user interface display that includes elements for digitally submitting user information, (2) trigger a user device (e.g., included as part of UE 102) to present the user interface display via a display element of the user device, and/or (3) receive user information as user input submitted to the elements presented within the user interface display. Environmental control engine 200 may trigger the user device to present the user interface display in a variety of digital contexts (e.g., as part of a registration process, as input to a health tracking application, as input to a smart home control application, as input to a sleep management application, etc.).

In some embodiments, the user data may be derived and/or mined from stored user data within an associated or third-party cloud. For example, environmental control engine 200 can be associated with a cloud, which can store collected network traffic and/or collected user data for the user in an associated account of the user. Thus, in some embodiments, Step 304 can involve querying the cloud for information about the user, which can be based on a criteria that can include, but is not limited to, a time, date, activity, event, other collected user data, and the like, or some combination thereof.

In some embodiments, the collected user data in Step 304 can be stored in database 108 in association with an identifier (ID) of a user, an ID of the location and/or an ID of an account of the user/location. In some examples, database 108 may also include information relating (e.g., relevant) to the user data. For example, in embodiments in which a user's user data includes a location of a home of the user, database 108 may include data relating to the location (e.g., a population of a city encompassing the location, a measure of pollution associated with the location, etc.). In some examples, environmental control engine 200 may collect data, according to the systems and features described herein (e.g., at Steps 302 and 304 and elsewhere), for multiple users (e.g., unrelated users engaging with different instances of UE 102 within different instances of network 104). In these examples, database 108 may represent a repository of user data maintained for each of the multiple users.

In Step 306, environmental control engine 200 can analyze the data (e.g., the user data) collected at Step 304. According to some embodiments, environmental control engine 200 can implement any type of known or to be known computational analysis technique, algorithm, mechanism or technology to analyze the collected user data from Step 306.

In some embodiments, environmental control engine 200 may include a computerized model such as a specific trained artificial intelligence/machine learning model (AI/ML), a particular machine learning model architecture, a particular machine learning model type (e.g., convolutional neural network (CNN), recurrent neural network (RNN), autoencoder, support vector machine (SVM), and the like), or any other known or to be known type of AI/ML model and/or any suitable combination thereof.

In some embodiments, environmental control engine 200 may be configured to utilize one or more AI/ML techniques chosen from, but not limited to, computer vision, feature vector analysis, decision trees, boosting, support-vector machines, neural networks, nearest neighbor algorithms, Naive Bayes, bagging, random forests, logistic regression, and the like. By way of a non-limiting example, engine 200 can implement an XGBoost algorithm for regression and/or classification to analyze the user data, as discussed herein.

According to some embodiments, the AI/ML computational analysis algorithms implemented can be applied and/or executed in a time-based manner, in that collected user data for specific time periods can be allocated to such time periods so as to determine patterns of activity (or non-activity) according to a criteria. For example, environmental control engine 200 can execute a Bayesian determination for a predetermined time span, at preset intervals (e.g., a 24-hour time span and/or 8-hour time span, for example), so as to segment the day according to applicable patterns, which can be leveraged to determine, derive, extract or otherwise activities/non-activities (e.g., in/around a location). In another example, the patterns can correspond to portions of the day that correspond to nighttime, sleep time, morning time, and/or waketime (e.g., which can be based on sunrise and/or sunset at the user's location and/or a window of time designated by a user).

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
c. apply the trained model to process the newly received input data,
f. optionally and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

In Step 308, based on the analysis from Step 306, environmental control engine 200 can generate (e.g., determine or receive), for a user based on the data collected at step 304, a user bedtime schedule. The bedtime schedule may refer to any type or form of organized data relating to a user's routine prior to sleeping (e.g., a routine performed in preparation for sleep). The user bedtime schedule may include a variety of data points. In some examples, the user bedtime schedule may include an action and/or a set of actions (e.g., a list of actions) that the user is expected (e.g., predicted) to perform in preparation for sleep. These actions may include any type or form of action performed in preparation for sleep (e.g., brushing teeth, turning on a particular light and/or a particular lamp, locking a door, taking out the trash, boiling water in a tea kettle, performing some type of exercise such as going for a walk or stretching, talking on the phone, scrolling and/or streaming content on a user device, taking a shower or a bath, checking email, opening the refrigerator to cat a snack, etc.). In some examples, the actions may include a movement-based action such as a movement pattern (e.g., walking from one room to another to perform a certain task such as feeding the dog, entering a room in which a user sleeps, etc.).

In certain examples in which the user bedtime schedule includes one or more user actions, the user bedtime schedule may include timing information for the one or more user actions. The timing information may be structured in a variety of ways. As one example, the timing information may include an average time at which an action is performed (e.g., on days for which data is collected). Additionally or alternatively, the timing information may include an expected (e.g., predicted) time at which an action is to be performed. This expected time may be calculated in a variety of ways (e.g., based on the average time, calculated as a determined offset from a starting time determined for the bedtime schedule, etc.). In some examples, the expected time for an action may be received as an output from a trained model such as a neural network. In some examples, the timing information may include a single time (e.g., a time at which a bedtime routine is expected to begin and/or a designated time before an anticipated bedtime).

In some examples, the user bedtime schedule may include a list of devices that control a stimulus (e.g., in the user's home environment) during a time associated with the bedtime schedule. Such devices may include any device (e.g., such as one or more of the devices discussed in connection with step 302). Specific examples of such a device include, without limitation, a device that controls a light source (e.g., a smart light switch, a smart shade or curtain, a smart light, and/or a device that controls one or more of these devices), a device that controls a temperature in the user's home environment (e.g., a smart thermostat, smart ceiling fan, and/or a device that controls one or more of these devices), a device that controls sound in the user's home environment (e.g., a smart speaker and/or a device that controls a smart speaker), a device that controls the presenting of content via a screen in the user's home environment (e.g., a user device such as a smart phone and/or a table and/or a television and/or a device that controls one or more of these devices), a device that controls Internet traffic in the user's home environment and/or that presents content via the Internet traffic (e.g., a router and/or a user device), an adjustable bed that controls a bed configuration, etc. In some examples, this list of devices may be generated using one or more of the computational analysis techniques, algorithms, mechanisms or technologies described above at Step 306.

In certain examples, one or more of the devices identified in the bedtime schedule may be associated with one or more of the user actions identified in the bedtime schedule. A device may be associated with an action in a variety of ways. In some examples, the device may be used to perform the action (e.g., a smart kettle may be used to boil water, a smart toothbrush may be used to brush teeth, and/or a tablet may be used to scroll a digital newsfeed). Additionally or alternatively, the device may facilitate (e.g., enable) the action (e.g., a ceiling light may emit a light that enables the user to safely walk across the room and/or a smart speaker may play soothing music that assists the user in falling asleep).

In some such examples, engine 200 can determine, for a device in the list of devices, an adjustment to a stimulus, controlled by the device, associated with an improved measure of sleep (e.g., one or more of the measures of sleep discussed previously). The adjustment to the stimulus may represent any type or form of adjustment that may be controlled by a device. Examples of such adjustments include, without limitation, an adjustment to a temperature (e.g., an ambient temperature in the home environment), an adjustment to a color temperature and/or a brightness of light, a sound adjustment (e.g., an adjustment to a kind of sound and/or a volume of sound), an adjustment to a bed configuration, a screen time limit, an Internet traffic limit, a television limit, an adjustment to content presented on a screen and/or received via Internet traffic (e.g., an adjustment limiting or restricting any content and/or a particular type of content such as content from a particular source), etc.

Engine 200 can determine that the adjustment is associated with an improved measure of sleep in a variety of ways. In one embodiment, the adjustment may have been determined to improve sleep in the user specifically. For example, engine 200 may have determined that the adjustment correlates with an improved measure of sleep for the user based on observational data collected and analyzed for the user (e.g., as described in Steps 304 and 306). Additionally or alternatively, the adjustment may have been determined as an improver of sleep quality on a population level. In some such embodiments, engine 200 may have identified the adjustment based on an analysis of aggregated data collected from many users (e.g., using the techniques and processes described at Steps 302, 304, 306, and 308). In some examples, engine 200 can identify the adjustment in a data store of device-controllable stimuli associated with improved sleep quality (e.g., with an improved measure of sleep).

In some examples, the adjustment may have been determined to improve (or predicted to improve) a measure of the user's sleep quality when applied as part of the user's bedtime schedule. For example, engine 200 may determine that the adjustment improved a measure of the user's sleep when applied to a stimulus presented to the user in associated to a particular action of the bedtime schedule (e.g., while a particular action was performed by the user) and/or may predict the same. As a specific example, engine 200 may have determined that playing white noise from a bathroom speaker while the user was brushing the user's teeth on a particular night or set of nights improved a measure of sleep for the user that night or set of nights. Additionally or alternatively, engine 200 may have determined that an adjustment to a stimulus improved (or is predicted to improve) a measure of the user's sleep when applied at a specified time (e.g., a designated number of minutes before the user's bedtime). As a specific example, engine 200 may have determined that changing a spectrum of light emitted from one or more light sources (e.g., any light source controllable by engine 200) one hour before a user's bedtime improved a measure of the user's sleep. Other examples of stimuli that may be determined to improve a measure of sleep quality include, without limitation, screen time limits, Internet traffic limits, television limits, preferred temperatures in the home environment, lighting instructions, etc.

In some examples, the user bedtime schedule may include a user bedtime (e.g., a time at which the user begins attempting to fall asleep). This user bedtime may be a user-identified time (e.g., a bedtime submitted to an interface by the user) and/or a time that is automatically identified based on the data collected and analyzed at Steps 304 and 306). In some examples, the user bedtime may be consistent (e.g., for each day of the week and/or month of the year). In other examples, the user bedtime may be variable. As a specific example, the user bedtime schedule may include (e.g., predict) a first user bedtime for a user for weekdays and a second user bedtime for weekends.

In addition, or as an alternative, to generating the user bedtime schedule described above, engine 200 can, based on the analysis from Step 306 of the data collected at step 304, generate (e.g., determine or receive) a user waketime schedule. The waketime schedule may refer to any type or form of organized data relating to a user's routine upon waking up (e.g., a waketime period routine in preparation for the day after sleeping). The waketime period (e.g., a temporal period) corresponding to the waketime schedule may be defined in a variety of ways. In some examples, the waketime period may be defined as a designated length of time (e.g., one hour) after a morning event (e.g., waking and/or getting out of bed). In other examples, the waketime period may be define as a length of time that is initiated by a first user action (e.g., waking up and/or getting out of bed) and terminated by a second user action (e.g., brushing teeth, leaving the kitchen, leaving the house, etc.).

The user waketime schedule may include a variety of data points. In some examples, the user waketime schedule may include an action and/or a set of actions (e.g., a list of actions) that the user is expected (e.g., predicted) to perform upon waking up. Examples of such user actions include, without limitation, any of the actions described in connection with bedtime schedule, a morning exercise routine (e.g., a morning jog or morning yoga), etc. As with the bedtime schedule, the morning schedule may include timing information (e.g., for the actions) which may be structured in a variety of ways (e.g., including one ore more of the structures described above in connection with the nighttime schedule).

As with the bedtime schedule, the waketime schedule may include a list of devices that control a stimulus (e.g., in the user's home environment) during a time associated with the waketime schedule. Such devices may include any device, including any of the devices described above in connection with the nighttime schedule.

As with the bedtime schedule, one or more of the devices identified in the waketime schedule may be associated with one or more of the user actions identified in the waketime schedule. A device may be associated with a user action in a variety of ways (e.g., as described above in connection with the nighttime schedule).

In some examples, engine 200 can determine, for a device in the list of devices identified in the waketime schedule, an adjustment to a stimulus, controlled by the device, associated with an improved measure of wakefulness and/or an improved measure of sleep on a night following the waketime schedule. The measure of wakefulness may refer to any type or form of metric (e.g., that may be collected by a sensor and/or received as user input from a user) including, without limitation, a subjective sense of being awake, a biometric indication of alertness, etc. In some examples, the measure of wakefulness may refer to a measure (e.g., a measure of productivity) collected later in the day following the waketime schedule. The adjustments may represent any type or form of adjustments that can be controlled by a device (e.g., one or more of the adjustments described previously in connection with the nighttime schedule). Engine 200 can determine that an adjustment is associated with an improved measure of wakefulness in a variety of ways. In some examples, engine 200 can make this determination using one or more of the techniques described above in connection with the nighttime schedule (e.g., measuring improved sleep in some examples and/or measuring improved wakefulness in place of or in addition to measuring improved sleep in other examples).

In some examples, the user waketime schedule may include a user waketime (e.g., a time at which the user wakes up and/or get out of bed). This may be a user-identified time (e.g., a time submitted to an interface) and/or a time that is automatically identified based on the data collected and analyzed (e.g., as described at Steps 304 and 306). As with the user bedtime, the user waketime may be consistent (e.g., across each day, week, or month) or variable.

In Step 310, environmental control engine 200 can store the information (e.g., generated at step 308 such as the information included in the bedtime schedule and/or the waketime schedule) in database 108, in a similar manner as discussed above. According to some embodiments, Step 310 can involve creating a data structure associated with each determined user action, each determined device, and/or each determined stimulus, whereby each data structure can be stored in a proper storage location associated with an identifier, as discussed above. In some embodiments, the data structure for a pattern can be relational, in that the events of a pattern can be sequentially ordered, and/or weighted so that the order corresponds to events with more or less activity.

In some embodiments, the structure of the data structure for the user actions, devices, and/or device stimuli can enable a more computationally efficient (e.g., faster) search of the user actions, devices, and/or device stimuli. In some embodiments, the data structures can be, but are not limited to, files, arrays, lists, binary, heaps, hashes, tables, trees, and the like, and/or any other type of known or to be known tangible, storable digital asset, item and/or object.

According to some embodiments, the user data can be identified and analyzed (e.g., at steps 302, 304, and 306) in a raw format, whereby upon a determination of the bedtime schedule or the waketime schedule, the data can be compiled into refined data (e.g., a format capable of being stored in and read from database 108). Thus, in some embodiments, Step 310 can involve the creation and/or modification (e.g., transformation) of the user data (e.g., the bedtime routine data) into a storable format.

In some embodiments, environmental control engine 200 can control the digital environment for the user for sleep optimization and/or wakefulness optimization by instructing a device to apply an adjustment to a stimulus (i.e., a stimulus that is associated with an improved sleep quality and/or wakefulness), as discussed below in relation to Process 400 of FIG. 4.

Turning to FIG. 4, Process 400 provides non-limiting example embodiments for the deployment and/or implementation of the disclosed sleep environment optimization framework. According to some embodiments, Step 402 can be performed by identification module 202 of environmental control engine 200 and Step 404 can be performed by output module 208. Accordingly, Steps 402 and 404 can be performed via engine 200 (and the corresponding modules) executing the AI/ML models in a similar manner as discussed above.

According to some embodiments, Process 400 begins with Step 402. In Step 402 (e.g., based on the sensor data collected and analyzed in the steps of FIG. 3), engine 200 can determine a bedtime schedule initiation (e.g., can determine that the user has initiated the user's bedtime schedule in preparation for sleep). In some embodiments, a bedtime schedule initiation can correspond to a range of time related to the user's predicted bedtime (e.g., a predetermined number such as 30 minutes). For example, determining the bedtime schedule initiation can involve identifying when the user is predicted to go to sleep, and the initiation time can be a designated time (e.g., 30 minutes) prior. Additionally or alternatively, engine 200 can determine a bedtime schedule initiation in response to detecting a triggering event (e.g., an event determined based on data collected by one or more of the devices described at step 302). As a specific example, engine 200 can determine a bedtime schedule initiation in response to determining that the user is brushing the user's teeth or has left a certain area in a home environment (e.g., a dining room). Additional examples of triggering events include, without limitation, turning out a particular light or set of lights (e.g., all the lights on a particular floor), entering a particular area within the home environment, turning on a particular light or set of lights, etc. In some examples, the triggering event may be defined in the bedtime schedule (e.g., as a first user action performed in the schedule). In some examples, the initiation may be determined by a combination of triggers (e.g., a triggering event occurring within a designated time frame).

In certain examples, engine 200 can detect one or more actions (e.g., actions delineated in the bedtime schedule) being performed by the user (e.g., during a period of time corresponding to the bedtime schedule). In some examples, these detected actions may be used (e.g., as input to the computerized model described at Steps 304, 306, and 308 in FIG. 3) to continually update the user's bedtime schedule (e.g., as described at Steps 304, 306, and 308 in FIG. 3).

Additionally or alternatively, in Step 402 engine 200 can determine a waketime schedule initiation. In some examples, the waketime schedule initiation can correspond to a designated time (e.g., a time for which the user has set an alarm or has indicated that the user would like to wake up) and/or a range of time (e.g., a designated time prior to a time for which the user has set an alarm or has indicated that the user would like to wake up). In some examples, engine 200 can determine the waketime schedule initiation (e.g., can determine that the waketime schedule is initiated) in response to detecting a triggering event (e.g., an event determined based on data collected by one or more of the devices described at step 302). As a specific example, engine 200 can determine a waketime schedule initiation in response to determining that the user has woken up, that the user has gotten out of bed, that the user has turned on a light, etc. In some examples, the initiation may be determined by a combination of triggers (e.g., a triggering event occurring within a designated time frame). As a specific example, a user waking up after 6 am may be configured to indicate that the waketime routine is initiated but a user waking up prior to 6 am may not.

As with the bedtime schedule, engine 200 can detect one or more actions (e.g., actions delineated in the waketime schedule) being performed by the user (e.g., during a period of time corresponding to the waketime schedule), which may be used to continually update the user's waketime schedule (e.g., as described at Steps 304, 306, and 308 in FIG. 3).

At Step 404, engine 200 can instruct a device (e.g., one of the devices in the bedtime schedule described at Step 308) to apply an adjustment associated with an improved sleep quality (e.g., as discussed at Step 308) to a stimulus controlled by a device identified in the bedtime schedule. Engine 200 may instruct the device to apply the adjustment in response to a variety of events. In some examples in which the device is associated with an action identified in the bedtime schedule, engine 200 can instruct the device to apply the adjustment in response to determining that the user is performing the action. Relatedly, engine 200 can instruct the device to apply the adjustment at a particular time in response to determining that the user is predicted to perform the action at the particular time. In such examples, the instruction may be triggered in a variety of ways. For example, the instruction may be triggered in response to determining the bedtime initiation as described in Step 402. As another example, the bedtime schedule may identify a series of actions performed in a particular order and the instruction may be triggered in response to determining that an additional action that comes before the action in the particular order has been performed by the user.

Additionally or alternatively at Step 404, engine 200 can instruct a device (e.g., one of the devices in the waketime schedule described at Step 308) to apply an adjustment associated with an improved wakefulness (e.g., as discussed at Step 308) to a stimulus controlled by a device identified in the waketime schedule. Engine 200 may instruct the device identified in the waketime schedule to apply the adjustment in response to a variety of events. In some examples in which the device is associated with an action identified in the waketime schedule, engine 200 can instruct the device to apply the adjustment in response to determining that the user is performing the action. As a specific example, engine 200 can instruct a mobile device to adjust a color temperature of light emitted from a bathroom light in response to determining that the user has entered the bathroom and/or has flipped on a light switch corresponding to the bathroom light. Relatedly, engine 200 can instruct the device to apply the adjustment at a particular time (e.g., in response to determining that the user is predicted to perform the action at the particular time). In some examples, the instruction may be triggered in response to determining the waketime initiation as described in Step 402. In one embodiment, the waketime schedule may identify a series of actions performed in a particular order and the instruction to apply the adjustment may be triggered in response to determining that an additional action that comes before the action in the particular order has been performed by the user.

According to some embodiments, a user and/or location can have a dedicated engine 200 model so that the sleep and/or wakefulness protocols discussed herein can be specific to the events and patterns learned and detected for the user and/or at that location. In some embodiments, the model can be specific for a user or set of users (e.g., users that live at a certain location (e.g., a house).

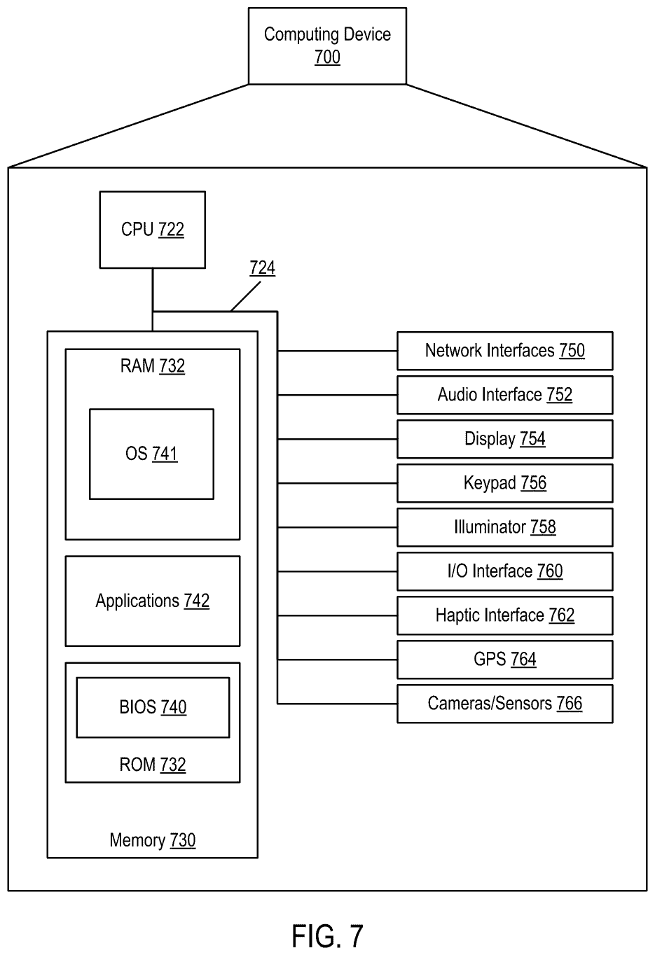

FIG. 7 is a schematic diagram illustrating a client device showing an example embodiment of a client device that may be used within the present disclosure. Client device 700 may include many more or less components than those shown in FIG. 7. However, the components shown are sufficient to disclose an illustrative embodiment for implementing the present disclosure. Client device 700 may represent, for example, UE 102 discussed above at least in relation to FIG. 1.

As shown in the figure, in some embodiments, Client device 700 includes a processing unit (CPU) 722 in communication with a mass memory 730 via a bus 724. Client device 700 also includes a power supply 726, one or more network interfaces 750, an audio interface 752, a display 754, a keypad 756, an illuminator 758, an input/output interface 760, a haptic interface 762, an optional global positioning systems (GPS) receiver 764 and a camera(s) or other optical, thermal or electromagnetic sensors 766. Device 700 can include one camera/sensor 766, or a plurality of cameras/sensors 766, as understood by those of skill in the art. For example, such sensors can include, but are not limited to, a photoplethysmogram (PPG) sensor, electrocardiogram (ECG) sensor, accelerometer, gyroscope, proximity sensor, and the like. Power supply 726 provides power to Client device 700.

Client device 700 may optionally communicate with a base station (not shown), or directly with another computing device. In some embodiments, network interface 750 is sometimes known as a transceiver, transceiving device, or network interface card (NIC).

Audio interface 752 is arranged to produce and receive audio signals such as the sound of a human voice in some embodiments. Display 754 may be a liquid crystal display (LCD), gas plasma, light emitting diode (LED), or any other type of display used with a computing device. Display 754 may also include a touch sensitive screen arranged to receive input from an object such as a stylus or a digit from a human hand.

Keypad 756 may include any input device arranged to receive input from a user. Illuminator 758 may provide a status indication and/or provide light.

Client device 700 also includes input/output interface 760 for communicating with external. Input/output interface 760 can utilize one or more communication technologies, such as USB, infrared, Bluetooth™, or the like in some embodiments. Haptic interface 762 is arranged to provide tactile feedback to a user of the client device.

Optional GPS transceiver 764 can determine the physical coordinates of Client device 700 on the surface of the Earth, which typically outputs a location as latitude and longitude values. GPS transceiver 764 can also employ other geo-positioning mechanisms, including, but not limited to, triangulation, assisted GPS (AGPS), E-OTD, CI, SAI, ETA, BSS or the like, to further determine the physical location of client device 700 on the surface of the Earth. In one embodiment, however, Client device may through other components, provide other information that may be employed to determine a physical location of the device, including for example, a MAC address, Internet Protocol (IP) address, or the like.

Mass memory 730 includes a RAM 732, a ROM 734, and other storage means. Mass memory 730 illustrates another example of computer storage media for storage of information such as computer readable instructions, data structures, program modules or other data. Mass memory 730 stores a basic input/output system (“BIOS”) 740 for controlling low-level operation of Client device 700. The mass memory also stores an operating system 741 for controlling the operation of Client device 700.

Memory 730 further includes one or more data stores, which can be utilized by Client device 700 to store, among other things, applications 742 and/or other information or data. For example, data stores may be employed to store information that describes various capabilities of Client device 700. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 700.

Applications 742 may include computer executable instructions which, when executed by Client device 700, transmit, receive, and/or otherwise process audio, video, images, and enable telecommunication with a server and/or another user of another client device. Applications 742 may further include a client that is configured to send, to receive, and/or to otherwise process gaming, goods/services and/or other forms of data, messages and content hosted and provided by the platform associated with engine 200 and its affiliates.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A computer-implemented method comprising:
generating, for a user based on data collected by one or more sensors in a home environment of the user, a user bedtime schedule comprising an action that the user is predicted to perform in preparation for sleep;
identifying a device associated with the action and an adjustment to a stimulus controlled by the device that is associated with an improved measure of sleep; and
in response to determining that the user is performing the action at a moment in time in preparation for sleep, or that the user is predicted to perform the action in preparation for sleep at the moment in time, causing the device to apply the adjustment to the stimulus at the moment in time, such that the home environment is modified based on the adjustment to the stimulus.

2. The method of claim 1, further comprising triggering the one or more sensors to collect the data.

3. The method of claim 1, wherein:
the method further comprises inputting the data collected by the one or more sensors to a computerized model; and
identifying the device and the adjustment associated with the improved measure of sleep comprises receiving at least one of the device or the adjustment as an output from the computerized model.

4. The method of claim 1, wherein generating the bedtime schedule comprises receiving the bedtime schedule as an output from a computerized model.

5. The method of claim 1, wherein:
the device comprises or controls a light source; and
the stimulus comprises a light emitted from the light source with an adjustable color temperature or brightness.

6. The method of claim 1, wherein:
the device controls an adjustable temperature in the home environment or controls an additional device that controls the adjustable temperature; and
the stimulus comprises the adjustable temperature.

7. The method of claim 1, wherein:
the device controls Internet traffic within the home environment or presents content received via the Internet traffic; and
the stimulus comprises the content received via the Internet traffic.

8. The method of claim 7, wherein the content comprises any content received from via the Internet traffic or particular content received via the Internet traffic.

9. The method of claim 1, further comprising:
generating, for the user based on the data collected by the one or more sensors in the home environment of the user, a user waketime schedule comprising a time at which the user is expected to wake up;
identifying an adjustment to a stimulus controlled by a device that is associated with improved wakefulness; and
in response to identifying the adjustment associated with improved wakefulness, instructing the device that controls the adjustment associated with improved wakefulness to apply the adjustment associated with improved wakefulness at the time at which the user is predicted to wake up.

10. The method of claim 9, wherein;
the waketime schedule further comprises an additional action that that user is expected to perform at an additional moment in time after waking up;
the method further comprises identifying a device associated with the additional action and an adjustment to a stimulus, controlled by the device associated with the additional action, that is associated with improved wakefulness; and
the method further comprises, in response to determining that the user is performing the additional action at the additional moment in time, or that the user is predicted to perform the additional action at the additional moment in time, instructing the device associated with the additional action to apply the adjustment associated with improved wakefulness at the additional moment in time.

11. The method of claim 1, wherein the one or more sensors comprise sensor capabilities for tracking movements and information about the user at the home environment.

12. A system comprising:
a physical processor; and
physical memory comprising computer-executable instructions that, when executed by the physical processor, cause the physical processor to:
generate, for a user based on data collected by one or more sensors in a home environment of the user, a user bedtime schedule comprising an action that the user is predicted to perform in preparation for sleep;
identify a device associated with the action and an adjustment to a stimulus controlled by the device that is associated with an improved measure of sleep; and
in response to determining that the user is performing the action at a moment in time in preparation for sleep, or that the user is predicted to perform the action in preparation for sleep at the moment in time, causing the device to apply the adjustment to the stimulus at the moment in time, such that the home environment is modified based on the adjustment to the stimulus.

13. The system of claim 12, wherein the computer-executable instructions further cause the physical processor to trigger the one or more sensors to collect the data.

14. The system of claim 12, wherein:
the computer-executable instructions further cause the physical processor to input the data collected by the one or more sensors to a computerized model; and
identifying the device and the adjustment associated with the improved measure of sleep comprises receiving at least one of the device or the adjustment as an output from the computerized model.

15. The system of claim 12, wherein generating the bedtime schedule comprises receiving the bedtime schedule as an output from a computerized model.

16. The system of claim 12, wherein:
the device comprises or controls a light source; and
the stimulus comprises a light emitted from the light source with an adjustable color temperature or brightness.

17. The system of claim 12, wherein:
the device controls an adjustable temperature in the home environment or controls an additional device that controls the adjustable temperature; and
the stimulus comprises the adjustable temperature.

18. The system of claim 12, wherein:
the device controls Internet traffic within the home environment or presents content received via the Internet traffic; and
the stimulus comprises the content received via the Internet traffic.

19. The system of claim 18, wherein the content comprises any content received from via the Internet traffic or particular content received via the Internet traffic.

20. A non-transitory computer-readable storage medium tangibly encoded with computer-executable instructions that, when executed by a device, perform a method comprising:
generating, for a user based on data collected by one or more sensors in a home environment of the user, a user bedtime schedule comprising an action that the user is predicted to perform in preparation for sleep;
identifying a device associated with the action and an adjustment to a stimulus controlled by the device that is associated with an improved measure of sleep; and
in response to determining that the user is performing the action at a moment in time in preparation for sleep, or that the user is predicted to perform the action in preparation for sleep at the moment in time, causing the device to apply the adjustment to the stimulus at the moment in time, such that the home environment is modified based on the adjustment to the stimulus.
